(19) United States
(12) Patent Application Publication
(10) Pub. No.: US 2019/0118002 A1
(43) Pub. Date: Apr. 25, 2019

(54) CALORIMETER

(71) Applicant: Sun Nuclear Corporation, Melbourne, FL (US)

(72) Inventor: Charles ROSENWALD, Melbourne, FL (US)

(73) Assignee: Sun Nuclear Corporation, Melbourne, FL (US)

(21) Appl. No.: 16/164,637

(22) Filed: Oct. 18, 2018

Related U.S. Application Data
(60) Provisional application No. 62/575,267, filed on Oct. 20, 2017.

(51) Int. Cl. A61N 5/10; G01K 17/04; G01N 25/20

(52) U.S. Cl. CPC A61N 5/1075; G01K 1/20; G01N 25/20; G01K 17/04

(57) ABSTRACT

A calorimeter includes a core, a conductive body at least partially enclosing the core, and at least two conductive elements coupled to the conductive body to directly heat the conductive body primarily by way of electrical current passing through the conductive body and causing resistive heating of the conductive body.

The current application claims priority under 35 U.S.C. § 119 to U.S. Provisional Patent Application No. 62/575,267 filed Oct. 20, 2017 and entitled “CALORIMETER,” the contents of which are hereby incorporated by reference in its entirety.

### BACKGROUND

Calibration and quality control of radiation treatment delivery devices used to administer radiation therapy can include measuring the radiation output of the radiation treatment delivery device. Diagnostic devices placed in the path of radiation generated by the radiation treatment delivery device can provide measurements of the output of the radiation treatment delivery device independent of the radiation treatment delivery device itself. By comparing the radiation output to an expected value, a calibration of the radiation treatment delivery device can be established or the radiation treatment delivery device can be adjusted to provide a desired output.

### SUMMARY

A calorimeter and methods for its manufacture and use are disclosed. Some implementations may include a core, a conductive body at least partially enclosing the core, and at least two conductive elements coupled to the conductive body to directly heat the conductive body primarily by way of electrical current passing through the conductive body and causing resistive heating of the conductive body.

In some variations, at least two conductive elements may include a first wire and a second wire electrically coupled to the conductive body to allow the electrical current to run from the first wire through the conductive body to the second wire and cause the resistive heating of the conductive body. The conductive elements may extend into a wall of the conductive body and may be electrically coupled to the conductive body.

In other variations, multiple conductive bodies may at least partially enclose the core. In some variations, each of the plurality of conductive bodies may be coupled to two or more conductive elements to resistively heat each of the plurality of conductive bodies. The conductive bodies may include a jacket at least partially enclosing the core, and a shield at least partially enclosing the jacket.

In yet other variations, the calorimeter may further include at least one of: a gap between at least two of the plurality of conductive bodies or between the conductive body and the core. The gap may be a vacuum gap or can contain an insulator. Implementations can include insulator being air or an aerogel.

In some variations, the core may be graphite or the conductive body can be graphite.

In an interrelated aspect, a calorimeter may include a core, a conductive body at least partially enclosing the core, a first conductive element coupled to the conductive body at a first location, and a second conductive element coupled to the conductive body at a second location separated from the first location to form a current path through a portion of the conductive body for directly heating the conductive body by resistive heating.

In some variations, the first conductive element may include a first wire and the second conductive element may include a second wire, the first wire and the second wire electrically coupled to the conductive body to allow electrical current to run from the first wire through the conductive body to the second wire and cause the resistive heating of the conductive body. In other variations, the first location and the second location may be at opposing locations on a diameter of the conductive body, at opposing locations on a perimeter of the conductive body, or at opposing locations on a length of the conductive body. Implementations can also include the portion that contains the current path having at least one of a diameter, a perimeter, or a length of the conductive body.

In other variations, the first and second conductive elements may extend into a wall of the conductive body and may be electrically coupled to the conductive body.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating an exemplary calorimeter in accordance with certain aspects of the present disclosure.

FIG. 2 is a diagram illustrating a sectional view of an exemplary core, conductive bodies, and gaps between the conductive bodies in accordance with certain aspects of the present disclosure.

FIG. 3 is a diagram illustrating an exploded view of the exemplary core, conductive bodies, and insulators in the gaps between the conductive bodies, in accordance with certain aspects of the present disclosure.

FIG. 4 is a diagram illustrating a perspective view of an exemplary conductive body and conductive elements in accordance with certain aspects of the present disclosure.

FIG. 5 is a diagram illustrating an exploded view of the exemplary conductive body and conductive elements shown in FIG. 4, in accordance with certain aspects of the present disclosure.

FIG. 6 is a diagram illustrating a sectional view of the exemplary core, conductive bodies, gaps, and conductive elements coupled to multiple conductive bodies in accordance with certain aspects of the present disclosure.

FIG. 7 is a diagram illustrating an exemplary method of determining an absorbed amount of radiation by the core in accordance with certain aspects of the present disclosure.

### DETAILED DESCRIPTION

A calorimeter can be used to determine information about a process by measuring a change in temperature of a portion of the calorimeter. For example, a material in the calorimeter can be exposed to radiation, such as that emitted from a radiation beam of a radiation treatment delivery device. The material, which can be part of a specific portion of the calorimeter, referred to herein as the “core” of the calorimeter, can absorb some portion of the radiation. The energy of the absorbed radiation can cause the temperature of the core to increase. In one example, when the amount and type of material making up the core is known (e.g., graphite), then based on the change in temperature of the core from absorbing radiation, the amount of radiation reaching the core can be determined.

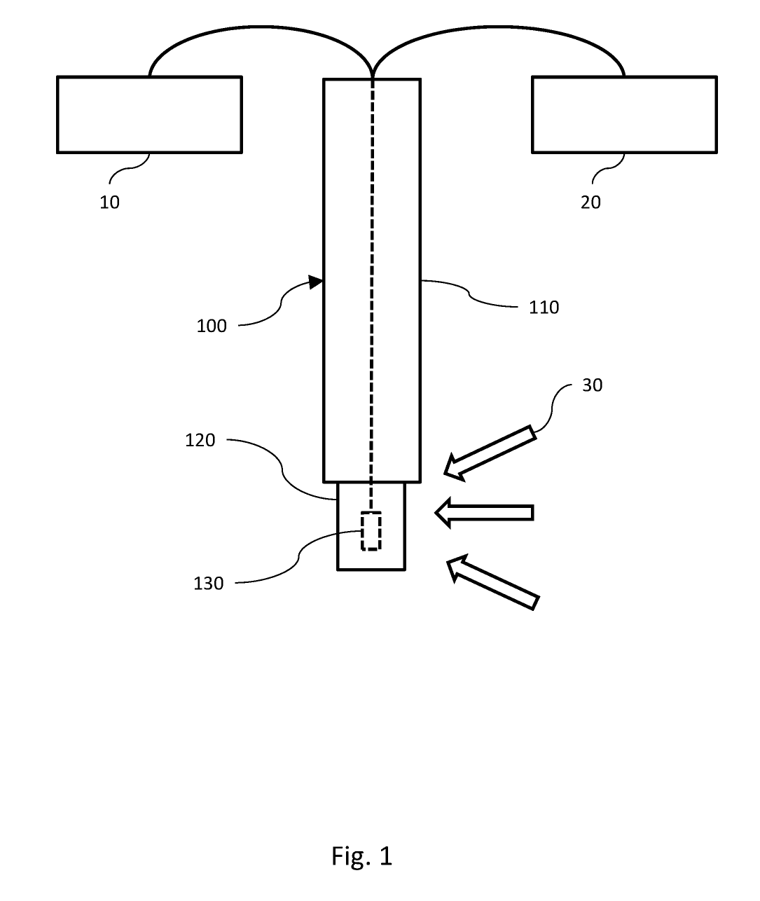

FIG. 1 is a diagram illustrating an exemplary calorimeter 100 in accordance with certain aspects of the present disclosure. In this example, calorimeter 100 is shown as having probe body 110 and probe tip 120 containing core 130. Calorimeter 100 can also be connected to controller 10 and/or data acquisition system 20.

Probe tip 120 can intercept and absorb radiation 30 to measure radiation at a particular location, in particular, at the location of core 130. In some implementations, core 130 can be contained wholly or partially within probe tip 120. In other implementations, calorimeter 100 need not be a “probe.” For example, calorimeter 100 can include only the portion labeled as probe tip 120. In such implementations, probe tip 120 can be positioned at the desired location by other means.

Optionally, a probe body 110 can be elongate and used to position probe tip 120 at the desired location to intercept radiation 30. Probe body 110 can contain wiring or other hardware required for the operation of calorimeter 100. As shown in the example of FIG. 1, probe body 110 can act as a conduit for connections from core 130 to controller 10 and/or data acquisition system 20.

Calorimeter 100 can also be operably connected to one or more other devices or computing systems for controlling the operation of calorimeter 100 and acquiring measurement data. Controller 10 can be operated by a user or by a computing system to, for example, regulate the powering of one or more components in calorimeter 100, supply power to heaters or heated elements in calorimeter 100 (as described further herein), receive feedback from sensing devices, thermistors, thermocouples, current monitors, etc. in calorimeter 100. In other implementations, a data acquisition system 20 can be connected to calorimeter 100. Data acquisition system 20 can receive signals, electrical impulses, or data from components within calorimeter 100. Information received at data acquisition system 20 can be processed at data acquisition system 20 or sent to other computing devices or hardware components.

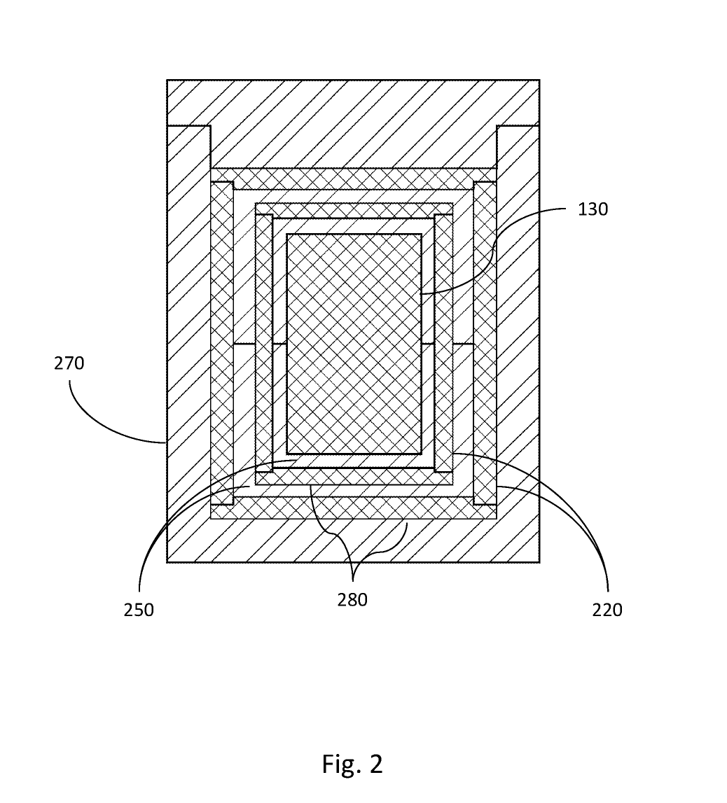

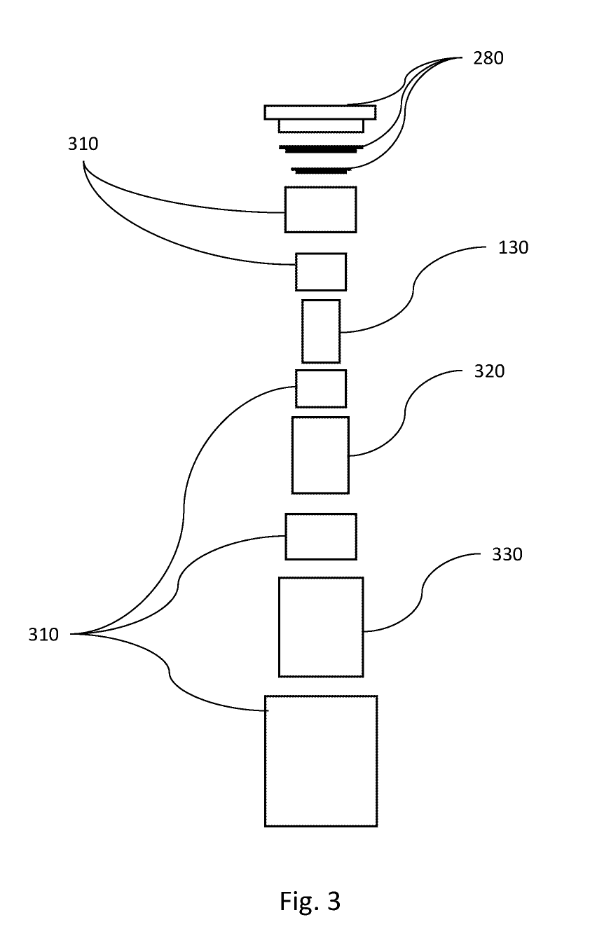

FIG. 2 is a diagram illustrating a sectional view of an exemplary core 130, conductive bodies 220, and gaps 250 between the conductive bodies in accordance with certain aspects of the present disclosure. FIG. 3 is a diagram illustrating an exploded view of the exemplary core 130, conductive bodies 220, and insulators 310 in the gaps 250 between the conductive bodies shown in FIG. 2, in accordance with certain aspects of the present disclosure.

In some implementations, calorimeter 100 can include a conductive body 220 at least partially enclosing core 130. Conductive body 220 can be a shell or other enclosure to isolate core 130 from external temperature changes and/or to conduct electricity for heating itself and its surroundings.

As shown in FIG. 2, some implementations can include multiple conductive bodies 220 at least partially enclosing core 130. For example, as shown in FIG. 3, a conductive body 220 may be referred to as jacket 320, which at least partially encloses core 130. Another conductive body 220 may be referred to as a shield 330, which at least partially encloses jacket 320. Though the example of FIG. 2 shows two conductive bodies 220 enclosing core 130, there can be any number of conductive bodies arranged or layered to enclose, or partially enclose, core 130. The assembly of conductive bodies 220 can also include one or more caps 280 to enclose openings in conductive bodies 220.

Some implementations, as shown in FIG. 2, can include a gap 250 between conductive bodies 220, between conductive body 220 and core 130, or both. In other implementations, adjacent conductive bodies 220 do not need to be spaced by gaps 250. For example, there may be no gap 250 between a jacket 320 and a shield 330.

In some implementations, gap 250 can be a vacuum gap. In other implementations, gap 250 can comprise an insulator. The insulator can be, for example, air, aerogel, or the like.

In another implementation, calorimeter 100 can include an outer layer 270 that surrounds core 130 and/or conductive bodies 220. Outer layer 270 can provide additional thermal isolation from the outside environment. Outer layer 270 can be, for example, an insulator such as aerogel, a plastic housing, or any other suitable material. Outer layer 270 can also provide electrical insulation for the outermost conductive body 220 (e.g., shield 330) to prevent shock or unwanted electrical discharge when a current is applied through the outermost conductive body 220.

As shown in FIGS. 2-4, calorimeter 100 can be of generally cylindrical construction. However, in other implementations, core 130, and any of the conducting bodies 220, can be spherical, cylindrical, pyramidal, polyhedral, with, for example, circular, ellipsoidal, rectangular, square, or hexagonal cross-sections. Core 130 and/or any or all of conducting bodies 220 can be constructed of any suitable conducting material, for example, graphite, aluminum, steel, copper, or the like. It is not necessary that the entire conducting body be constructed of any single particular material. For example, there can be portions of conducting body 220 that are different materials, including combinations of insulators and/or conductors, so long as the conducting body may be heated by resistive heating.

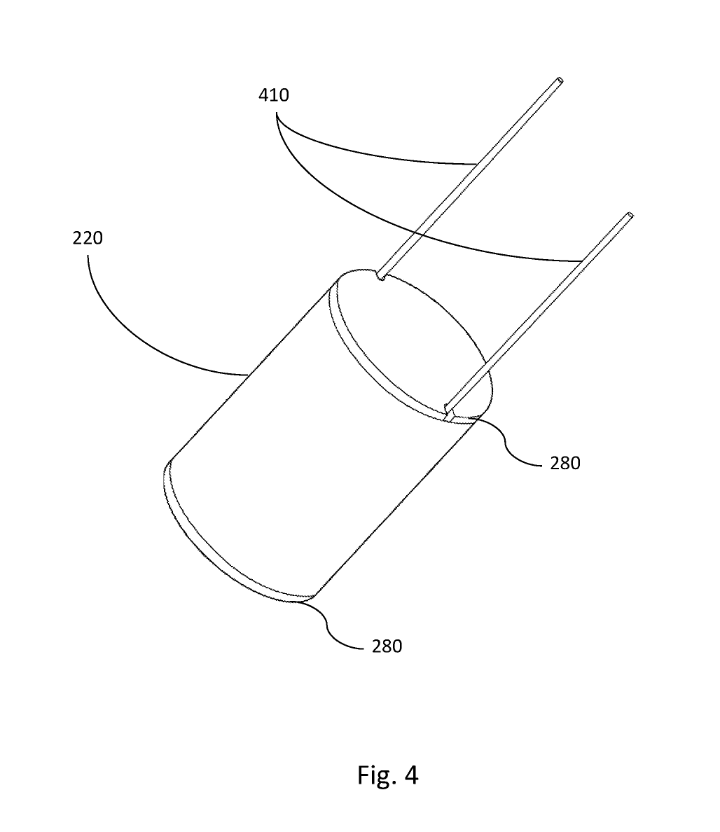

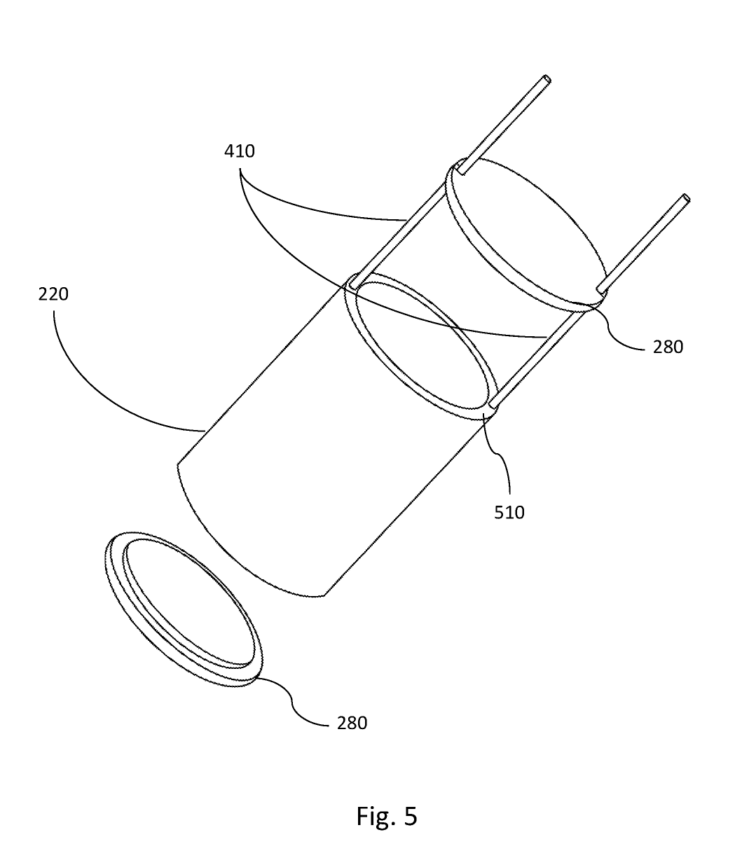

FIG. 4 is a diagram illustrating a perspective view of an exemplary conductive body 220 and conductive elements 410 in accordance with certain aspects of the present disclosure. FIG. 5 is a diagram illustrating an exploded view of the exemplary conductive body 220 and conductive elements 410 shown in FIG. 4, in accordance with certain aspects of the present disclosure.

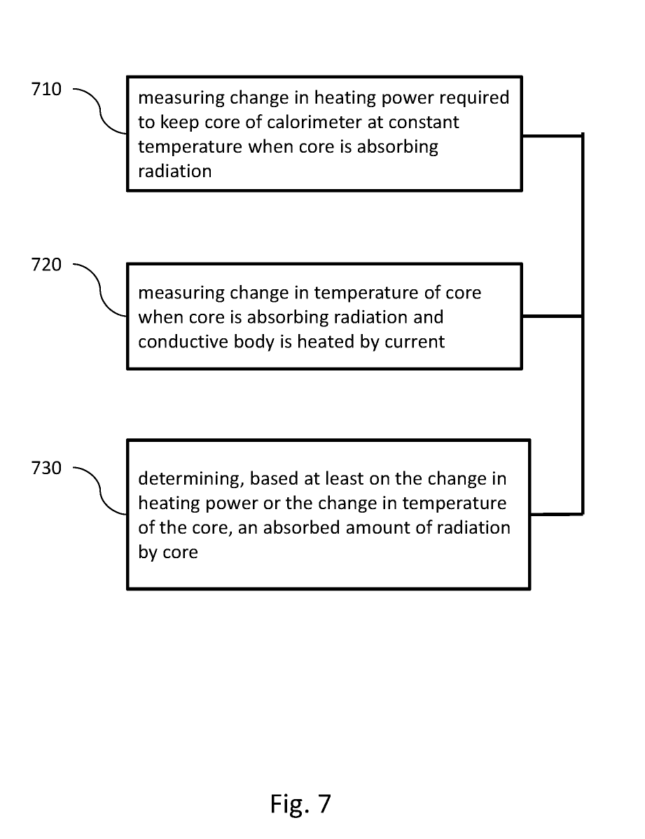

In some implementations, one or more conductive bodies 220 can be heated to stabilize the temperature of core 130 against outside temperature changes so that the temperature changes in core 130 are due only to radiation absorption and can be measured. In other implementations, changes in the heating power required to keep core 130 at a constant temperature can be measured to determine an amount of absorbed radiation by core 130. Implementations of such methods are further described with reference to FIG. 7.

The temperature of core 130 may be regulated by direct heating of one or more conductive bodies 220 surrounding core 130. In some implementations, a conductive body 220 can be heated primarily by way of passing current through conductive body 220 and causing resistive heating of conductive body 220. The current can be delivered to conductive body 220 by, for example, at least two conductive elements 410 coupled to conductive body 220. Conductive elements 410 can be, for example, wires. As used herein, “resistive heating” generally refers to the heating that occurs in conductive body 220 by the flow of an electrical current through conductive body 220. The presence of resistive heating does not explicitly exclude other sources of heating, for example, a temperature change due to thermal conduction from nearby components of calorimeter 100. For example, conductive body 220 can be heated directly by resistive heating but can also receive heat from another nearby conductive body 220, core 130, or other heat sources. In other implementations, core 130 can itself be directly heated by attaching conductive elements 410 similarly to those shown attached to conductive body 220.

In some implementations, such as that illustrated in FIGS. 4 and 5, conductive elements 410 can include a first wire and second wire electrically coupled to conductive body 220 to allow electrical current to run from the first wire through conductive body 220 to the second wire and cause resistive heating of conductive body 220. Conductive elements 410 can be coupled to conductive body 220 by, for example, extending conductive elements 410 into wall 510 of conductive body 220. Conductive elements 410 may be electrically coupled to conductive body 220, for example, by welding, epoxying, soldering, attaching with fasteners, or the like.

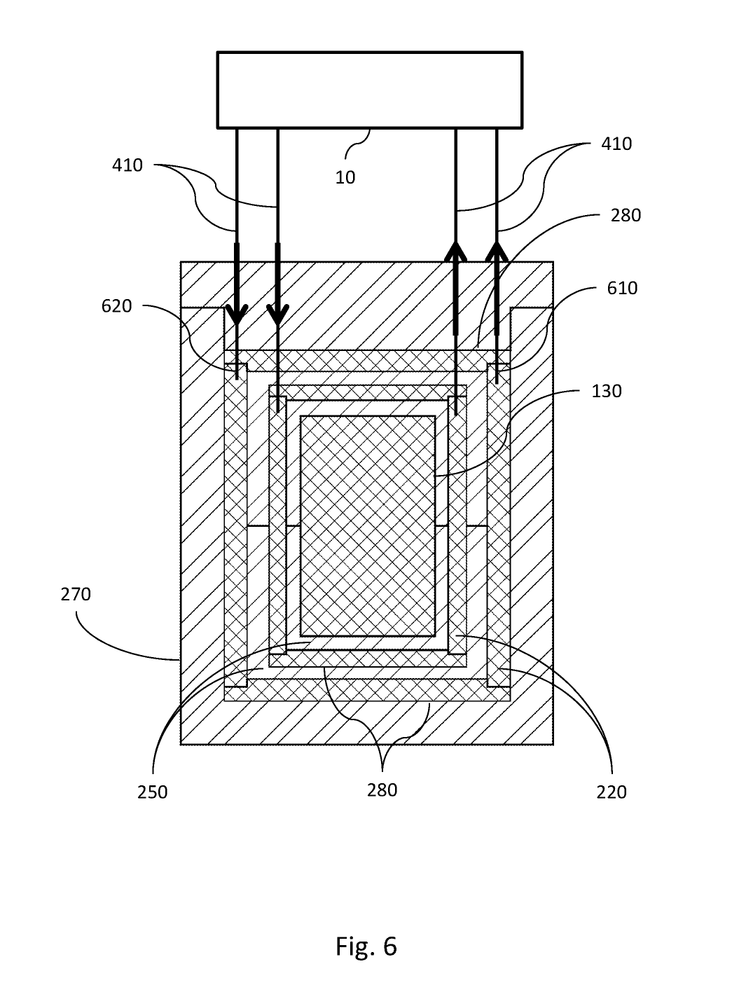

FIG. 6 is a diagram illustrating a sectional view of the exemplary core 130, conductive bodies 220, gaps 250, and conductive elements 410 coupled to multiple conductive bodies 220 in accordance with certain aspects of the present disclosure. In implementations such as that illustrated in FIG. 6, each of the conductive bodies 220 can be coupled to at least two conductive elements 410 to resistively heat each of conductive bodies 220. Each conductive body 220 may be connected to an independent pair of conducting elements as shown in FIG. 6 to provide independent heating or can be connected in series to provide a single current through at least two of conductive bodies 220.

In some implementations, as shown in FIG. 6, first conductive element 420 (e.g., a first wire) can be coupled to conductive body 220 at first location 610. Second conductive element 430 (e.g., a second wire) can be coupled to conductive body 220 at second location 620 separated from first location 610 to form current path through a portion of conductive body 220 for directly heating conductive body 220 by resistive heating. FIG. 6 illustrates one example of first location 610 and second location 620. In some implementations, first location 610 and second location 620 can be on opposite sides of conductive body 220. For example, first location 610 and second location 620 can be at opposing locations on a diameter, perimeter, or length of conductive body 220.

When controller 10 includes, or is otherwise coupled to, a power supply, then, for example, electrical current can be run through a first wire connected at first location 610, through conductive body 220, and out the second wire. As used herein, “current path” is defined to mean a path along which current can flow through conductive body 220. A current path can include, for example, the shortest or least resistive path between two conductive elements 410, but may also include other paths through conductive body 220 depending on the electrical properties of conductive body 220, locations where conductive elements 410 are attached, etc. In some implementations, the portion that contains current path can include at least one of the diameter, perimeter, or length of conductive body 220.

The example of FIG. 6 shows conductive elements 410 connected at opposing sides of one end of a conductive body 220. Similarly, first location 610 and second location 620 can be on opposite sides of conductive body 220. In implementations where conductive elements 410 are at opposing ends of conductive body 220, the electrical current may flow through a greater portion of conductive body 220.

FIG. 7 is a diagram illustrating an exemplary method of determining an absorbed amount of radiation 30 by core 130 in accordance with certain aspects of the present disclosure. The present disclosure contemplates several methods of determining an amount of absorbed radiation 30. One method, referred to herein as an isothermal mode of operation, can include, at 710, measuring a change in heating power required to keep core 130 of calorimeter 100 at a constant temperature when core 130 is absorbing radiation 30. For example, when core 130 is being heated by the absorption of energy from incoming radiation 30, less power (or current) may be required to keep the temperature of core 130 at a predefined temperature. In some implementations, the predefined temperature can be a temperature elevated by the heating to be above the ambient temperature surrounding calorimeter 100.

Another method, referred to herein as a quasi-adiabatic mode of operation, can include, at 720, measuring a change in temperature of core 130 when core 130 is absorbing radiation 30 and conductive body 220 is heated by the current. Here, where the heating power is held constant, the amount of radiation 30 absorbed by core 130 can be determined based in part on the change the temperature of core 130.

Given the two example methods above, a processor or computing system can then determine, at 730, based at least on the change in heating power or the change in temperature of core 130, an absorbed amount of radiation 30 by core 130.

There are numerous references herein to the measurement of temperatures and the control of the temperature of various elements (e.g., core 130 and/or conductive bodies 220). In any of the implementations described herein, calorimeter 100 can include temperature sensors operatively connected to any of the components such as core 130 or conductive bodies 220. Temperature sensors can include, for example, thermistors, thermocouples, bimetallic strips, or the like. Data generated by the temperature sensors can be incorporated by a processor or software program to control (e.g., with a controller 10) electrical currents used for heating or to determine changes in temperature resulting from absorbed radiation 30.

The present disclosure contemplates that the calculations disclosed in the embodiments herein may be performed in a number of ways, applying the same concepts taught herein, and that such calculations are equivalent to the embodiments disclosed.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” (or “computer readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” (or “computer readable signal”) refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, computer programs and/or articles depending on the desired configuration. Any methods or the logic flows depicted in the accompanying figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Specifically, and by way of example, although the headings refer to a “Technical Field,” such claims should not be limited by the language chosen under this heading to describe the so-called technical field. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

What is claimed is:

1. A calorimeter comprising:
a core;
a conductive body at least partially enclosing the core; and
at least two conductive elements coupled to the conductive body to directly heat the conductive body primarily by way of electrical current passing through the conductive body and causing resistive heating of the conductive body.

2. The calorimeter of claim 1, wherein the at least two conductive elements include a first wire and a second wire electrically coupled to the conductive body to allow the electrical current to run from the first wire through the conductive body to the second wire and cause the resistive heating of the conductive body.

3. The calorimeter of claim 1, wherein the at least two conductive elements extend into a wall of the conductive body and are electrically coupled to the conductive body.

4. The calorimeter of claim 1, further comprising a plurality of conductive bodies at least partially enclosing the core.

5. The calorimeter of claim 4, wherein each of the plurality of conductive bodies are coupled to two or more conductive elements to resistively heat each of the plurality of conductive bodies.

6. The calorimeter of claim 4, the plurality of conductive bodies including a jacket at least partially enclosing the core, and a shield at least partially enclosing the jacket.

7. The calorimeter of claim 4, further comprising at least one of: a gap between at least two of the plurality of conductive bodies or between the conductive body and the core.

8. The calorimeter of claim 7, wherein the gap is a vacuum gap.

9. The calorimeter of claim 7, wherein the gap comprises an insulator.

10. The calorimeter of claim 9, wherein the insulator is air.

11. The calorimeter of claim 9, wherein the insulator is an aerogel.

12. The calorimeter of claim 1, wherein the core is graphite.

13. The calorimeter of claim 1, wherein the conductive body is graphite.

14. A calorimeter comprising:
a core;
a conductive body at least partially enclosing the core;
a first conductive element coupled to the conductive body at a first location; and
a second conductive element coupled to the conductive body at a second location separated from the first location to form a current path through a portion of the conductive body for directly heating the conductive body by resistive heating.

15. The calorimeter of claim 14, wherein the first conductive element includes a first wire and the second conductive element includes a second wire, the first wire and the second wire electrically coupled to the conductive body to allow electrical current to run from the first wire through the conductive body to the second wire and cause the resistive heating of the conductive body.

16. The calorimeter of claim 14, wherein the first location and the second location are at opposing locations on a diameter of the conductive body.

17. The calorimeter of claim 14, wherein the first location and the second location are at opposing locations on a perimeter of the conductive body.

18. The calorimeter of claim 14, wherein the first location and the second location are at opposing locations on a length of the conductive body.

19. The calorimeter of claim 14, wherein the portion that contains the current path includes at least one of a diameter, a perimeter, or a length of the conductive body.

20. The calorimeter of claim 14, wherein the first and second conductive elements extend into a wall of the conductive body and are electrically coupled to the conductive body.
